(19) United States
(12) Patent Application Publication
(10) Pub. No.: US 2019/0000629 A1
(43) Pub. Date: Jan. 3, 2019

(54) INTRAOPERATIVE CUSTOM VOID FILLING METHODS, SYSTEMS AND APPARATUSES

(71) Applicant: Biomet Manufacturing, LLC, Warsaw, IN (US)

(72) Inventor: Nathan A. Winslow, Warsaw, IN (US)

(21) Appl. No.: 16/020,168

(22) Filed: Jun. 27, 2018

Related U.S. Application Data
(60) Provisional application No. 62/526,073, filed on Jun. 28, 2017.

(51) Int. Cl. A61F 2/28; A61B 17/17; A61F 2/40; A61F 2/30; A61B 34/10

(52) U.S. Cl. CPC A61F 2/2846; A61B 17/1778; A61F 2/4081; A61F 2/30942; A61B 34/10; A61F 2002/2835; A61B 2017/00526; A61F 2310/00353; A61F 2310/00359; A61F 2002/30957; A61F 2002/30945; A61F 2002/30948; A61B 2034/108; A61F 2002/285

(57) ABSTRACT

Methods, systems and apparatuses for treating a defect in a bone that forms at least a portion of a joint of a patient are disclosed. According to one example, a system including a first prosthesis, a second prosthesis and an alignment guide is disclosed. The first prosthesis can comprise a formable material. The second prosthesis can have a stock construction and can be configured to be implanted in the joint on at least a portion of the first prosthesis once the first prosthesis is formed to take a shape of and substantially fill a volume of the defect. The alignment guide can be configured to couple to the second prosthesis to retain the second prosthesis in a desired position and a desired orientation in the joint on the at least the portion of the first prosthesis.

### CLAIM OF PRIORITY

This application claims the benefit of U.S. Provisional Patent Application Ser. No. 62/526,073, filed on Jun. 28, 2017, the benefit of priority of which is claimed hereby, and which is incorporated by reference herein in its entirety.

### FIELD

The present subject matter relates to an orthopedic devices, systems and methods, and specifically to combinations of devices that can be used to treat a defect in a patient's anatomy.

### BACKGROUND

In a healthy shoulder, the proximal humerus is generally ball-shaped, and articulates within a socket formed by the scapula, called the glenoid, to form the shoulder joint.

Patients may require joint repair in various anatomical areas to restore natural joint movement and reduce pain. Shoulder joint reconstruction may require repairing a defect in a shoulder joint such as a void in the glenoid resulting from severe wear.

Current methods for reconstructing the shoulder joint are sometimes not sufficiently accurate to reproduce the natural joint movement of the shoulder joint such as glenoid version without excessive time, trial and/or expense. One of the challenges when installing an implant in the glenoid relates to the positioning of the implant. Due to the presence of soft tissue such as ligaments, the positioning of the implant must be planned to replicate as much as possible the normal bio-mechanical movements of the humerus relative to the scapula. For patients with severe joint damage (e.g. as a result of a defect), obtaining a desired placement and orientation of the implant can be even more challenging.

Considering these and other challenges, patient specific implants and instrumentation (hereinafter “PSI”) has been used in some cases. PSI pertains to the creation of implants and instruments that are made specifically for the individual patient. PSI are typically manufactured from data using imagery to model bone geometry. Therefore, PSI have surfaces that may contact the bone in a predictable way as such contact surfaces are specifically manufactured to match the surface of a bone. It would be desirable to use PSI technology in shoulder surgery. However, PSI can be cost prohibitive, complex and/or too time consuming to implement for mass production.

By way of example, some digital reconstruction systems used can be used in cases of severe bone loss, such as to the glenoid. With digital reconstruction systems, a patient-matched implant is created to fill bone voids. This requires work to be done by engineers and other personnel at a dedicated manufacturing facility in advance of surgery, and requires a custom implant to be sent to the surgical facility prior to surgery.

### OVERVIEW

Examples of the present application are described in reference to the shoulder joint of a patient. However, it should be recognized that the techniques, methods, systems and prostheses described can be applicable to any anatomical feature including any joint of the patient. The anatomical feature can also include a non-joint portion of a bone or other anatomy of the patient (e.g., soft tissue) in some cases.

According to some examples of the present application, a combination of a first prosthesis, a second prosthesis and an alignment guide are disclosed. The first prosthesis can be a material configured to substantially take a shape of and substantially fill a volume of a defect in a bone of the patient. The second prosthesis can have a stock construction (i.e. can come in various predetermined standard sizes and can have a predetermined shape that is common to all the prostheses produced—these and other characteristics are not changed on a patient by patient basis in view of imaging data obtained using a medical imaging technique on a specific patient). According to one example, the second prosthesis can be implanted in the joint on at least a portion of the first prosthesis once the first prosthesis is formed to substantially take the shape of and substantially fill the volume of the defect. The alignment guide can be configured to couple to the second prosthesis to retain the second prosthesis in a desired position and a desired orientation in the joint on the at least the portion of the first prosthesis (e.g., while the first prosthesis cures).

According to the disclosed examples, one or both of the alignment guide and the first prosthesis can treat the defect in the bone and can allow a stock implant (the second prosthesis) to be provided with the desired position and the desired orientation. In this manner, the present disclosed devices, systems and methods can avoid the use of PSI for the second prosthesis, as the second prosthesis can be implanted in the desired position and the desired orientation despite being stock. As such, the additional costs and additional time that would otherwise be incurred had a PSI prosthesis been utilized (rather than the stock prosthesis) can be avoided.

To further illustrate the apparatuses, systems and methods disclosed herein, a non-limiting list of examples is provided here:

Example 1 is a system for treating a defect in a bone that forms at least a portion of a joint of a patient, the system can optionally include: a first prosthesis comprising a formable material; a second prosthesis having a stock construction, the second prosthesis configured to be implanted in the joint on at least a portion of the first prosthesis once the first prosthesis is formed to take a shape of and substantially fill a volume of the defect; and an alignment guide configured to couple to the second prosthesis to retain the second prosthesis in a desired position and a desired orientation in the joint on the at least the portion of the first prosthesis.

In Example 2, the subject matter of Example 1 can optionally include the first prosthesis comprises one or more of a bone graft, bone, and a synthetic bone putty.

In Example 3, the subject matter of any one or more of Examples 1-2 can optionally include the first prosthesis in combination with the alignment guide provide for the desired position and the desired orientation of the second prosthesis relative to the joint.

In Example 4, the subject matter of any one or more of Examples 1-3 can optionally include a first mold configured to form the first prosthesis, wherein the first mold is formed based on imaging data obtained using a medical imaging technique performed on the joint of the patient, and wherein the first mold includes a cavity that replicates the volume of the defect.

In Example 5, the subject matter of Example 4 can optionally include the first mold includes one or more of a surface and a second cavity configured to replicate an articular surface of the joint.

In Example 6, the subject matter of any one or more of Examples 1-5 can optionally include a resection guide configured to position a cutting tool at the joint to perform a resection to remove at least a portion of joint including the defect to create a void with a desired shape in the joint.

In Example 7, the subject matter of Example 6 can optionally include the first prosthesis is configured to substantially take a shape of the void either by insertion into the joint and the void or by being formed in a second mold having a cavity that replicates a volume of the void.

In Example 8, the subject matter of any one or more of Examples 1-7 can optionally include wherein the alignment guide has one of a stock construction or a patient specific construction.

In Example 9, the subject matter of Example 8 can optionally include wherein the patient specific construction includes a guide surface configured to nest and closely conform to a first surface such that the guide surface mates with the first surface when the alignment guide is in only one orientation.

In Example 10, the subject matter of Example 9 can optionally include wherein the first surface comprises at least one of a surface or an edge of the first mold or the second mold, a surface of the bone that forms the joint, a second surface of the bone that forms the joint the second surface external of the joint, and a surface of a second bone.

In Example 11, the subject matter of Example 10 can optionally include wherein the joint comprises a shoulder and the bone comprises a scapula, and wherein the defect is in a glenoid of the scapula but the first surface comprises a second portion of the scapula external of the glenoid.

Example 12 is an assembly for treating a defect in a bone that forms at least a portion of a joint of a patient, the assembly can optionally include: a first prosthesis comprising a formable material that is formed to take a shape of and substantially fill a volume of the defect; a second prosthesis having a stock construction, the second prosthesis implanted in the joint on at least a portion of the first prosthesis; and an alignment guide connected to the second prosthesis to retain the second prosthesis in a desired position and a desired orientation in the joint on the at least the portion of the first prosthesis.

In Example 13, the subject matter of Example 12 can optionally include wherein the first prosthesis comprises one or more of a bone graft, bone, and a synthetic bone putty.

In Example 14, the subject matter of any one or more of Examples 12-13 can optionally include wherein the first prosthesis in combination with the alignment guide provide for the desired position and the desired orientation of the second prosthesis.

In Example 15, the subject matter of any one or more of Examples 12-14 can optionally include wherein the first prosthesis substantially takes the shape of the defect either by insertion into the defect or by being formed in a mold prior to insertion into the joint.

In Example 16, the subject matter of Example 15 can optionally include wherein the mold is inserted into the joint and the alignment guide is connected to the mold.

In Example 17, the subject matter of any one or more of Examples 12-16 can optionally include wherein the alignment guide has one of a stock shape or a patient specific shape.

In Example 18, the subject matter of Example 17 can optionally include the patient specific shape includes a guide surface configured to nest and closely conform to a first surface such that the guide surface mates with the first surface when the alignment guide is in only one orientation.

In Example 19, the subject matter of Example 18 can optionally include the first surface comprises at least one of a surface or an edge of the mold, a surface of the bone that forms the joint, a second surface of the bone that forms the joint the second surface external of the joint, or a surface of a second bone.

Example 20 is a method of repairing a defect in a bone that forms at least a portion of a joint of a patient, the method can optionally include: receiving imaging data obtained using a medical imaging technique, the imaging data representing the joint of the patient; creating a three-dimensional model of the joint including the defect based on the imaging data; implanting a first prosthesis in the joint, the first prosthesis formable to take a shape of and substantially fill a volume of the defect; implanting a second prosthesis in the joint, the second prosthesis disposed on at least a portion of the first prosthesis; and coupling an alignment guide to the second prosthesis to retain the second prosthesis in a desired position and a desired orientation in the joint on the at least the portion of the first prosthesis.

In Example 21, the subject matter of Example 20 can optionally include forming a mold based upon the imaging data, the mold including a cavity that replicates the volume of the defect; and shaping the first prosthesis within the cavity.

In Example 22, the subject matter of any one or more of Examples 19-21 can optionally include providing a patient specific guide surface for the alignment guide based upon the imaging data, the guide surface configured to nest and closely conform to a first surface such that the guide surface mates with the first surface when the alignment guide is in only one orientation.

In Example 23, the subject matter of Example 22 can optionally include the first surface comprises at least one of a surface or an edge of the mold, a surface of the bone that forms the joint, a second surface of the bone that forms the joint the second surface external of the joint, or a surface of a second bone.

In Example 24, the subject matter of any one or more of Examples 20-23 can optionally include creating a three-dimensional model comprising a virtual resection guide based on the imaging data; fabricating a resection guide configured to guide a cutting tool in resecting the portion of the joint that includes the defect to create a void with a desired shape; and shaping the first prosthesis to substantially take a shape of the void either by insertion into the joint and the void or by being formed in a second mold having a cavity that replicates a volume of the void.

In Example 25, the apparatuses, system and/or method of any one or any combination of Examples 1-24 can optionally be configured such that all elements or options recited are available to use or select from.

These and other examples and features of the present apparatuses, systems and methods will be set forth in part in the following Detailed Description. This Overview is intended to provide non-limiting examples of the present subject matter—it is not intended to provide an exclusive or exhaustive explanation. The Detailed Description below is included to provide further information about the present systems and methods.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various examples discussed in the present document.

FIG. 1 is a perspective view illustrating use of a medical imaging technique, such as a computed tomography (CT scan), x-ray or a magnetic resonance imaging (MRI), to obtain imaging data representing a shoulder joint according to an example of the present application.

FIG. 2A is a three-dimensional (3D) model illustrating a perspective view of a scapula generated based on imaging data obtained using an imaging technique such as that illustrated in FIG. 1 according to an example of the present application.

FIG. 2B is a cross-sectional view of the scapula illustrated in the 3D model of FIG. 2A taken along line 2B-2B shown in FIG. 2A according to an example of the present application.

FIG. 3 is perspective view illustrating placement of both a first prosthesis in a defect in a glenoid of the scapula and a second prosthesis in the glenoid on at least a portion of the first prosthesis according to an example of the present application.

FIGS. 4A and 4B are perspective views illustrating a mold having a volume and one or more surfaces constructed from the imaging data in order to replicate the defect in the glenoid of FIG. 3, the mold configured to form the first prosthesis to fill the defect in the scapula as shown in FIG. 4B according to an example of the present application.

FIG. 5 shows a perspective view of an assembly including the first prosthesis and second prosthesis of FIG. 3, and further including an alignment guide coupled to the second prosthesis according to an example of the present application.

FIG. 6 is a highly schematic view of a second assembly according to another example of the present application.

FIG. 7 is a perspective view of a resection guide according to an example of the present application, the resection guide positioned on a glenoid to create a void in the glenoid according to an example of the present application.

FIG. 8 is a perspective view illustrating placement of a first prosthesis into the void in the glenoid created using the resection guide of FIG. 7 and additionally showing placement of a second prosthesis on at least a portion on the first prosthesis according to an example of the present application.

### DETAILED DESCRIPTION

Example apparatuses, systems and methods are described including those for treating a defect in a bone that forms at least a portion of a joint of a patient are disclosed. The disclosed instruments and prostheses are for use in shoulder joint replacement, shoulder resurfacing procedures and other procedures related to the shoulder joint or the various bones of the shoulder joint, including the scapula (specifically the glenoid) and adjacent shoulder bones. The present techniques can be applied to anatomic shoulder replacement and reverse shoulder replacement.

As previously discussed, according to some examples a combination of a first prosthesis, a second prosthesis and an alignment guide are disclosed. The alignment guide and/or the first prosthesis can be used with the second prosthesis, which can have a stock construction. According to the disclosed examples, one or both of the alignment guide and the first prosthesis can treat the defect in the bone and can allow the second prosthesis (of stock construction) to be provided with the desired position and the desired orientation. As such, the present disclosure can achieve additional cost and time savings as the second prosthesis need not be PSI. For example, the first prosthesis can accurately conform to the defect and substantially fill the defect to provide a continuous surface with the bone surface surrounding the defect. Additionally, the alignment guide can at least initially provide the desired position and the desired orientation for the second prosthesis. Thus, the natural movement of the shoulder joint, including glenoid version, may be reproduced.

According to some examples of the present disclosure and for various instruments and prostheses, PSI can be utilized. PSI can include, for example, molds, molding guides, cutting/resection guides or alignment guides. However, according to other examples various of the instruments and prostheses disclosed may not be PSI in nature but rather of stock or other construction. Indeed, the prostheses described in many of the examples provided comprise stock (conventional) prostheses. However, in other cases one or more of the prostheses can be PSI that can be prepared using computer-assisted image methods.

The term “PSI”, “custom-made” or “customized” are defined herein to apply to components, including tools (e.g., guides), molds, prostheses, portions or combinations thereof, which include geometric features, including surfaces, curves, or other lines that are made to closely conform as mirror-images or negatives to corresponding anatomical landmarks (features, surfaces, voids, etc.) of an individual patient's anatomy. In some examples, the geometric features, including surfaces, curves, or other lines can only mate with the corresponding anatomical landmark when the tool, mold, prosthesis, etc. is oriented in a single position. In the case of a PSI surface, such surface can be complementary to a corresponding surface of a patient's anatomy.

Imaging data used for PSI can be obtained or gathered during a pre-operative planning stage based on three-dimensional computer images of the corresponding anatomy reconstructed from image scans of the patient by computer imaging methods. Such images scans can be gathered using any known imaging modality including x-ray, ultrasound, magnetic resonance imaging (MRI) or a computed tomography (CT scan) of the individual patient's anatomy, for example. Further, PSI features can include guiding surfaces, guiding apertures, guiding slots, or other holes or openings. Such PSI features can be included in prostheses, alignment guides, resection guides, rasps or other instruments. These PSI features can be constructed (via use of three-dimensional modeling) to have positions, orientations, dimensions, shapes and/or define cutting planes and axes specific to the particular patient's anatomy including various anatomic or mechanical axes based on the computer-assisted pre-operative plan associated with the individual patient.

PSI should be contrasted with “stock”, “conventional”, “non-custom” or “off-the-shelf”, which are defined herein to apply to components (e.g., tools (such as guides), molds, prostheses) that come in various predetermined standard sizes and, in some cases, have a predetermined shape that is common to all the prostheses produced. These and other characteristics are not changed on a patient by patient basis in view of imaging data obtained using a medical imaging technique on a specific patient.

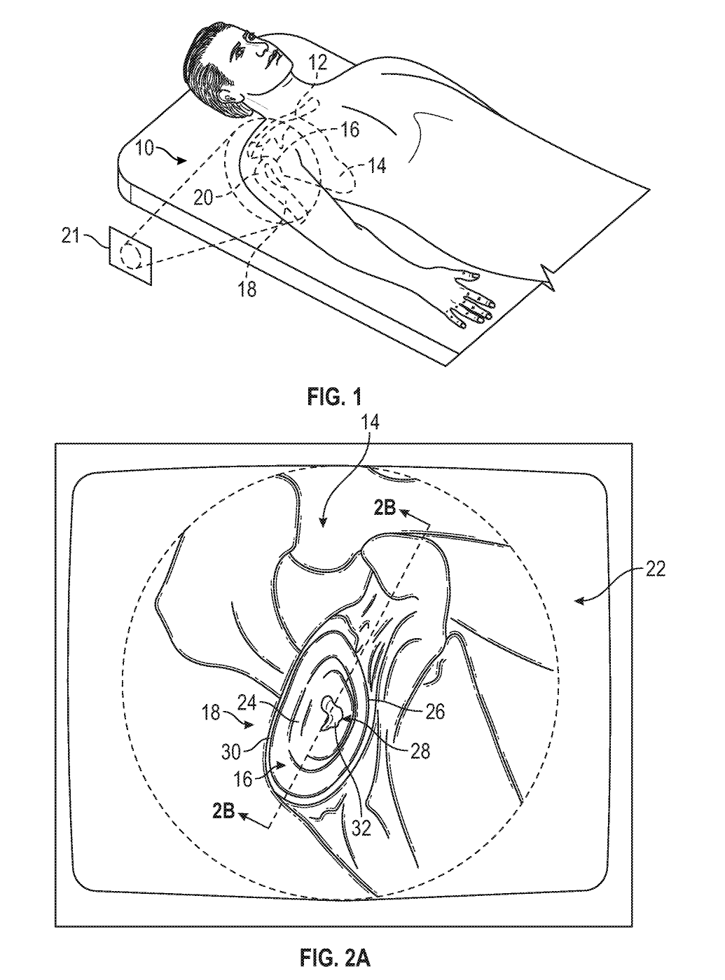

Referring to the drawings, and more particularly to FIG. 1, a medical imaging technique for obtaining imaging data representing a shoulder joint 10 is illustrated. The shoulder joint 10 includes a clavicle 12, a scapula 14 having a glenoid 16, and a humerus 18 having a head 20 that articulates within the glenoid 16. Medical imaging techniques that can be employed to obtain imaging data include, but are not limited to CT scan, MRI, x-ray, and ultrasound. An imaging device 21, such as a scanner, scans the shoulder joint 10 to generate imaging data representing the shoulder joint 10. The imaging data may represent two-dimensional or three-dimensional images of the shoulder joint 10. In one example, the imaging data represents two-dimensional sliced images of the shoulder joint 10 depicting cross-sections of the shoulder joint 10 that can be approximately a same distance apart from each other.

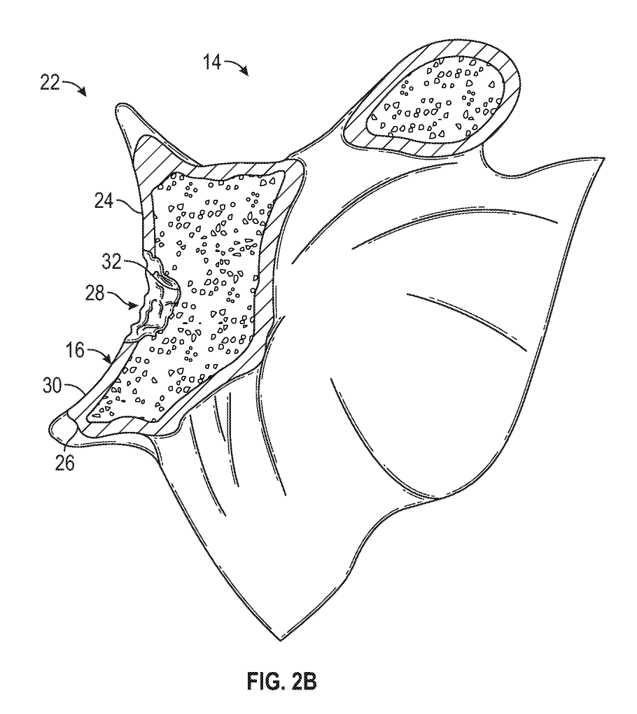

In FIGS. 2A and 2B, a three-dimensional model 22 of the scapula 14, including the glenoid fossa 16, is illustrated. The three-dimensional model 22 can be generated based on the imaging data obtained using a medical imaging technique such as illustrated in FIG. 1. The three-dimensional model 22 can be generated using software that generates three-dimensional models of an anatomical feature based on two-dimensional or three-dimensional imaging data corresponding to the anatomical feature. In one example, the three-dimensional model 22 can be generated using a process referred to as segmentation in which two-dimensional sliced images are converted into the three-dimensional model.

The scapula 14 can include an articular surface 24, which the humeral head 20 (FIG. 1) articulates relative to. Additionally, the scapula 14 can have a non-articular or perimeter surface 26 that surrounds the articular surface 24. The articular surface 24 forms the concave glenoid 16 (also referred to as the glenoid fossa) that receives the humeral head 20 (FIG. 1). In the example of FIGS. 2A and 2B, the glenoid 16 includes a defect 28, such as a void in the articular surface 24. This defect 28 can be the result of severe wear. The defect 28 can make it difficult to mount a conventional prosthesis, or indeed PSI prosthesis, on the glenoid 16 to achieve a proper position and orientation for the prosthesis for proper joint kinematics.

The articular surface 24 can include an outer surface 30. Such outer surface 30 can surround the defect 28 completely in some examples. The articular surface 24 can also include an irregular surface 32 at the location of the defect 28. The irregular surface 32 can define at least a portion of the volume of the defect 28. The irregular surface 32 can be recessed relative to the outer surface 30, as shown, which results in the void (depression) in the glenoid 16.

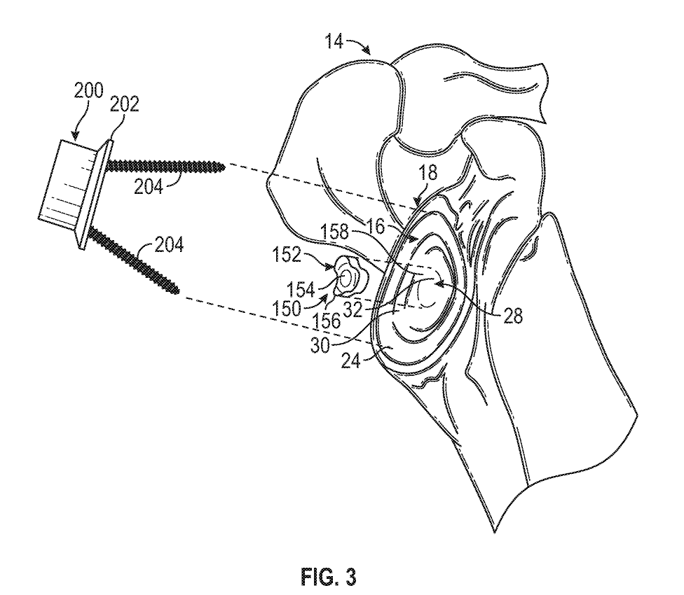

FIG. 3 shows placement of a first prosthesis 150 onto the defect 28 in the glenoid 16. Additionally, FIG. 3 shows placement of a second prosthesis 200 on the glenoid 16, such placement can be at least partially on the first prosthesis 150, which can be implanted prior to the second prosthesis 200. The second prosthesis 200 can include a body 202 and fasteners 204.

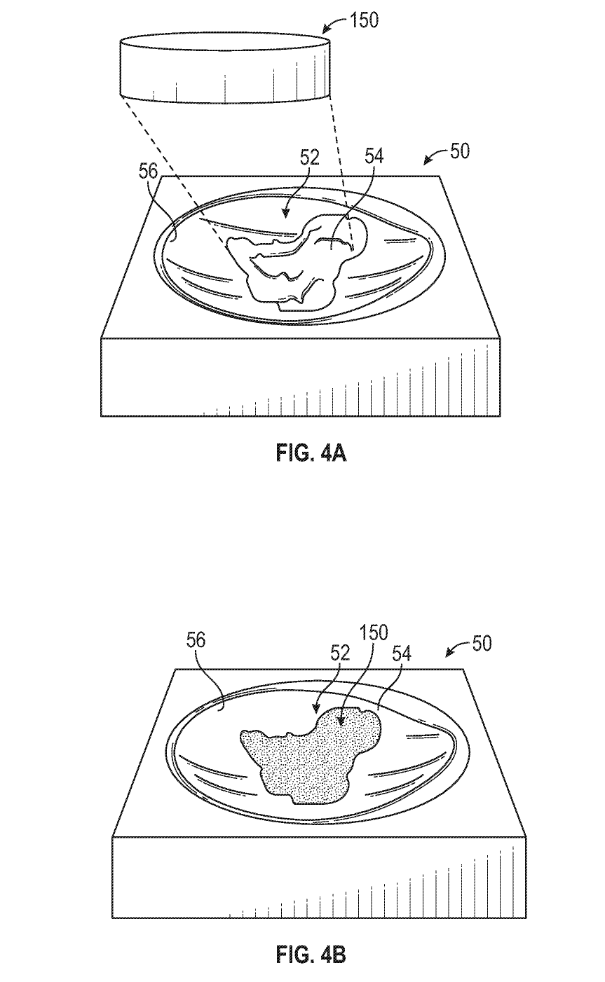

The first prosthesis 150 can comprise of a formable material so as to substantially take the shape of and substantially fill a volume of the defect 28. Thus, the first prosthesis 150 can be malleable and/or compressible according to some examples. More particularly, the first prosthesis 150 represents the final product after the formable material is shaped to match the surface of the defect 28 (e.g., the irregular surface 32 of FIGS. 2A and 2B) and to form a continuous surface with the surface surrounding the defect (e.g., the outer surface 30 of FIGS. 2A and 2B). The first prosthesis 150 can be formed using a mold such as the mold subsequently described in reference to of FIGS. 4A and 4B. Alternatively, the first prosthesis 150 can be shaped in vivo by being inserted into the defect 28 and can be formed by hand or tool.

According to some examples, the first prosthesis 150 can include at least two distinct surfaces—a first surface 152, which can nest with the irregular surface 32 and substantially fill the volume of the defect 28, and a second surface 154, which matches the surrounding articular surface. The first prosthesis 150 can be formed from many different formable materials such as, bone graft, autologous bone, allograft bone, xenograft bone, cortical bone, cancellous bone, and/or synthetic bone. In some cases, one or more of these can be provided in the form of a putty. Synthetic bone can include porous ceramic, such as Cerament™, having a density similar to that of cortical or cancellous bone. Alternatively, the first prosthesis 150 can be formed from a porous metal and a surgeon may insert growth factor and/or cancellous bone into the first prosthesis 150. As such, before placing the first prosthesis 150 onto the defect 28, a surgeon can apply growth factor to the first prosthesis 150 to facilitate formation of a bond between the first prosthesis 150 and the glenoid 16.

If a mold is utilized to pre-form the first prosthesis 150 prior to placing the first prosthesis 150 in the defect 28, the surgeon can align an outer perimeter 156 of the first prosthesis 150 with an outer perimeter 158 of the defect 28. The first surface 152 of the first prosthesis 150 can be designed to match the irregular surface 32 of the defect 28 such that there can be only one orientation for the first prosthesis 150 to mate with the defect 28. Once the first prosthesis 150 is in position, the second surface 154 can cooperate with the outer surface 30 surrounding the defect 28 to form a continuous articular surface. According to some examples, the surgeon can secure the first prosthesis 150 to the glenoid fossa 16 using fasteners or Kirschner wires (K-wires).

After implantation of the first prosthesis 150, the second prosthesis 200 can be installed. Thus, the second prosthesis 200 can be configured to be implanted in the joint once the first prosthesis 150 is formed to take the shape of and substantially fill the volume of the defect 28. In some examples, the second prosthesis 200 can be implanted on at least a portion of the first prosthesis 150 in addition to the glenoid 16. The second prosthesis 200 can include the body 202, which extends outward from the bone. The fasteners 204 or another type of fixation feature (e.g., pins, bone cement, K-wires, etc.) can retain the second prosthesis 200 in the joint. In some examples, the second prosthesis 200 can be retained in the joint by the first prosthesis 150 once the first prosthesis 150 has cured. The body 202 can be configured to connect to another implant (not shown) or another portion of the second prosthesis 200 (not shown). The shape of this portion can depend upon whether the second prosthesis 200 is being used as part an anatomic shoulder replacement or a reverse shoulder replacement, for example.

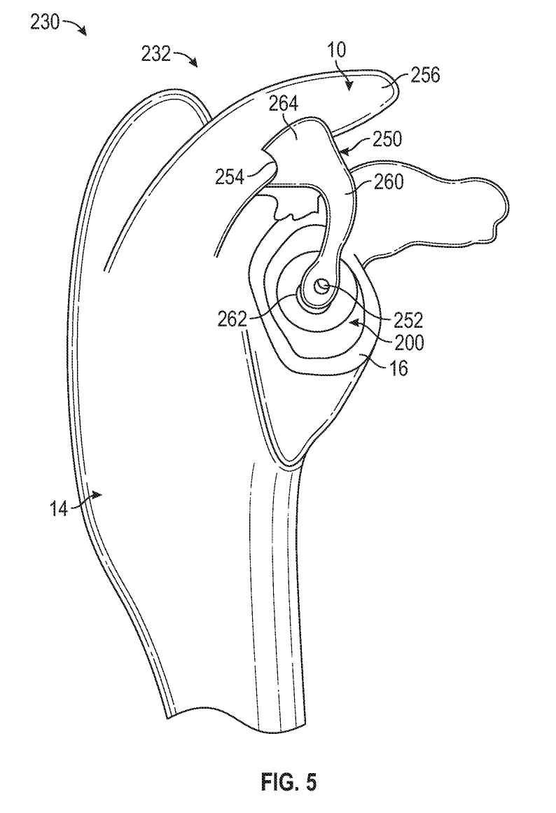

According to the example of FIG. 3, the second prosthesis 200 can have a stock construction. As will be discussed and illustrated subsequently in reference to FIG. 5, the second prosthesis 200 can be retained on the glenoid 16 and on at least a portion of the first implant 150 in a desired position and a desired orientation by an alignment guide.

FIGS. 4A and 4B show a mold 50 for creating the first prosthesis 150 (shown formed in FIG. 4B) for repairing the defect 28 in the glenoid 16 (shown in FIGS. 1, 2A and 2B). FIG. 4A shows the first prosthesis 150 prior to being formed in the mold 50. FIG. 4B shows the first prosthesis 150 after being formed in the mold 50.

The mold 50 can be created based on a three-dimensional model such as the three-dimensional model 22 of FIG. 2A. The mold 50 can be formed from a material such as a plastic, metal, other material or combination thereof, for example. In some examples, a manufacturing process such as machining, molding, and/or additive manufacturing can be used to create the mold 50.

As shown in FIG. 4A, the mold 50 can include a cavity 52 that replicates the volume of the defect 28. The cavity 52 also can include a first surface 54 that matches an actual surface of the defect 28 such as the irregular surface 32 of FIGS. 2A and 2B. The cavity 52 can include a second surface 56 that can be configured to match an outer surface of the anatomical feature surrounding the defect such as the outer surface 30 (FIGS. 2A and 2B). Thus, the second surface 56 can be configured to match a portion of the articular surface 24 of the glenoid 16 that surrounds the defect 28 in the glenoid 16.

The mold 50 can have an open design (i.e. have two parts), or can be a closed design with a riser that allows insertion of formable material 102 (shown in FIG. 4A), into the cavity 52. By having the first and second surfaces 54, 56 in the mold 50, the first prosthesis 150 can be precisely made to fit the defect volume, as well as match the articular surface.

According to some examples, a three-dimensional model of the mold (not shown) can be provided to a surgeon as part of a preoperative plan. Thus, the three-dimensional model can be provided to the surgeon before the mold 50 is formed. The surgeon can review the three-dimensional model and can provide direction via electronic input regarding the mold 50 construction. Additionally or alternatively, the surgeon can alter the three-dimensional model to communicate design changes via the electronic input. The mold 50 can then be formed based on the original three-dimensional model and/or the further input the surgeon provides.

As shown in FIG. 4B, the surgeon can use the mold 50 to form the first prosthesis 150 for repairing the defect, for example, by filling the three-dimensional volume of the mold 50. Once formed in the mold 50, the surgeon can remove the first prosthesis 150 therefrom and transfer the first prosthesis 150 to the joint to fill the defect 28 as previously shown and discussed in reference to FIG. 3.

FIG. 5 shows a system 230 that includes the combination of the first prosthesis 150 and the second prosthesis 200 as previously described in reference to FIG. 3 with the first prosthesis 150 and the second prosthesis 200 installed in the joint. In particular, FIG. 5 shows the patients' shoulder joint 10 including the glenoid 16 with the first prosthesis 150 and the second prosthesis 200 implanted thereon. FIG. 5 also shows additional features of the scapula 14. In the example of FIG. 5, the first prosthesis 150 is implanted into the defect 28 (not shown) to substantially fill the defect 28. As previously described in FIG. 3, the second prosthesis 200 can be implanted over the first prosthesis 150. In some cases, the second prosthesis 200 can extend to be implanted into other portions of the glenoid 16 and/or scapula 14.

As show in FIG. 5, the system 230 (shown as an assembly 232) can include the first prosthesis 150 and the second prosthesis 200 and additionally an alignment guide 250. According to the example of FIG. 5, the alignment guide 250 can be configured to couple to the second prosthesis 200 to retain the second prosthesis 200 in a desired position and a desired orientation in the joint on the at least the portion of the first prosthesis 150. Coupling of the alignment guide 250 with the second prosthesis 200 can be accomplished with a mechanical connection such as a pin 252. According to some examples, the coupling of the alignment guide 250 with the second prosthesis 200 can be temporary, and the alignment guide 250 can be decoupled from the second prosthesis 200 and removed from the patient once the second prosthesis 200 is securely implanted (e.g., by curing of the first prosthesis 150).

As previously discussed in reference to FIG. 3, according to some examples the second prosthesis 200 can have a stock construction. This can save costs and preoperative planning time v. if the second prosthesis were PSI. However, even with the second prosthesis 200 comprising a stock implant, the desired position and the desired orientation for the second prosthesis 200 can still be achieved with the aid of the alignment guide 250. Additionally or alternatively, the first prosthesis 150 in combination with the alignment guide 250 can provide for the desired position and the desired orientation of the second prosthesis 200 in the joint. The desired position and the desired orientation can be determined preoperatively, for example, with the aid of three-dimensional model 22 of FIGS. 2A and 2B. Furthermore, the desired position and the desired orientation for the second prosthesis 200 can be measured relative to any one or any combination of one or more articular surfaces of the joint, a center point of an anatomic feature (e.g., a center point of the glenoid), anatomic axes, anatomic features, a desired version (can be based upon a natural version), a desired inclination (can be based upon a natural inclination), or the like.

The alignment guide 250 can have one of a stock construction or a patient specific (i.e. PSI) construction. For example, the alignment guide 250 can include at least one guide surface 254 that is PSI in nature so as to be configured to nest and closely conform to a first surface (e.g., surface 256 of the scapula 14) such that the at least one guide surface 254 mates with the first surface 256 when the alignment guide 250 is in only one orientation. Thus, the alignment guide 250 can reference a surface or portion of the scapula 14 in order to position and orient the alignment guide 250 to achieve the desired position and the desired orientation for the second prosthesis 200.

More particularly, the alignment guide 250 can further include an arm 260 that extends away from a guide portion 262, and a head 264 that can be fixed to an end of the arm 260 opposite the guide portion 262. The head 264 can include the at least one guide surface 254 that can be PSI as discussed above. The guide portion 262 can be configured to connect to the second prosthesis 200 (e.g., via a pin 252). In some examples, the guide portion 262 and the arm 260 and/or the guide portion 262 and the head 264 can be moveably attached. For instance, in some embodiments, the alignment guide 200 can include a movable coupling, such as a pivoting joint, that moveably couples the guide portion 262 and the arm 260. Thus, in some examples the alignment guide 200 can move between a collapsed position and an extended position to engage with the surface 256 of the scapula 15 as shown.

The alignment guide 200 is illustrated as engaging the acromion of the scapula 14 external to the shoulder joint 12 in FIG. 5. However, in other examples the alignment guide 200 can contact any surface of the scapula 14 external of or within the shoulder joint 12 including one or more surfaces of features such as the scapular spine, a face and/or rim of the glenoid 16, the coronoid process, scapular notch, scapular fossa, scapular border, etc.

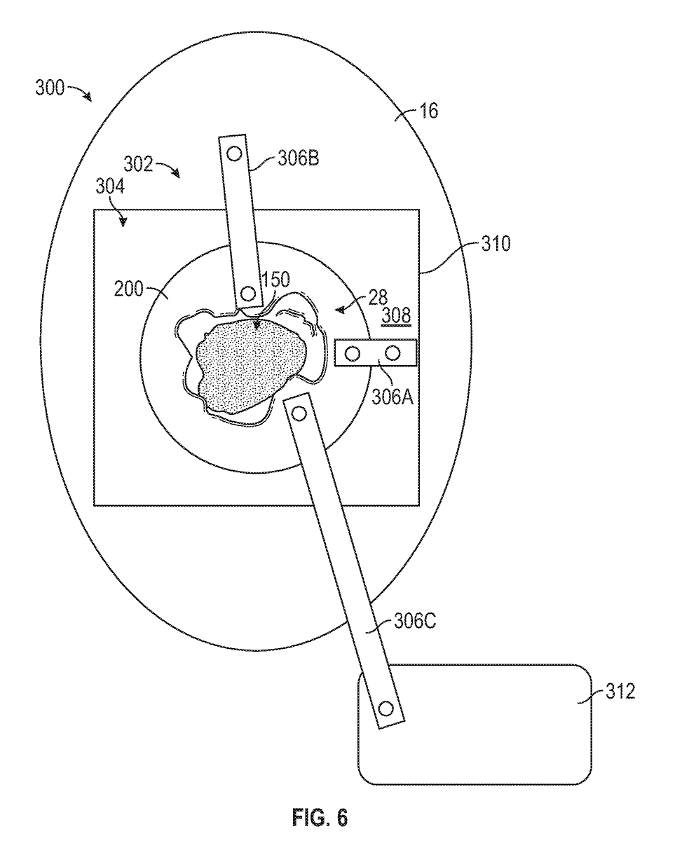

FIG. 6 shows a highly schematic view of a system 300 arranged as an assembly 302 on the glenoid 16 according to another example of the present application. The system 300 can include the first prosthesis 150 and the second prosthesis 200 as previously described. The first prosthesis 150 can be formed and implanted to fill the defect 28 in the glenoid 16 and the second prosthesis 200 can be implanted on at least a portion of the first prosthesis 150 as shown in FIG. 6. In FIG. 6, the first prosthesis 150 is illustrated as only partially filling the defect 28 in the glenoid 16 for demonstration purposes in order to also illustrate defect 28. It is understood that in most examples the first prosthesis 150 would entirely fill the defect 28. In some examples, the first prosthesis 150 could be allowed to cure prior to, during or after implantation of the second prosthesis 200 on the first prosthesis 150.

The example of FIG. 6 also includes a mold 304 that can be used to create a level edge/surface to seat the first prosthesis 150 into the defect 28. In some examples, the mold 304 can be removed prior to implantation of the second prosthesis 200. However, in other examples the mold 304 or portions of the mold 304 can be retained on the glenoid 16 upon implantation of the second prosthesis 200 on the glenoid 16 (and on at least a portion of the first prosthesis 150). The mold 304 can be constructed and designed in a manner previously discussed with regard to mold 50 of FIGS. 4A and 4B.

The example of FIG. 6 also includes alignment guides 306A, 306B and 306C configured to connect to at least a first surface (e.g., at least one of a surface or an edge of the mold, a surface of the bone that forms the joint, a second surface of the bone that forms the joint the second surface external of the joint, and a surface of a second bone). Two or more of the alignment guides 306A, 306B and 306C can be used together in some cases. According to other examples the alignment guides 306A, 306B and 306C can be used singularly as alternatives to one another. As shown in FIG. 6, the alignment guide 306A can connect to the second prosthesis 200 and can additionally extend to connect to at least one of a surface 308 or an edge 310 of the mold 304. The alignment guide 306B can connect to the second prosthesis 200 and can additionally extend to connect to a surface of the glenoid 16 (i.e. a surface of the bone that forms the joint). The alignment guide 306C can connect to the second prosthesis 200 and can additionally extend to connect to a second bone 312 (e.g., a clavicle, a rib, etc.).

The alignment guides 306A, 306B and 306C can be used to orient the second prosthesis 200, which can be a stock prosthesis, in a desired anatomical orientation. Additionally, the alignment guides 306A, 306B and 306C can optionally be used to retain the second prosthesis 200 only while the first prosthesis 150 is curing. After curing of the first prosthesis 150, the second prosthesis 200 can be coupled to the first prosthesis 150 in a desired anatomical orientation. In such instances, the alignment guides 306A, 306B and 306C can then be removed. Furthermore, although the second prosthesis 200 is illustrated as larger than the first prosthesis 150 in the example provided in FIG. 6, it is contemplated that in many instances the second prosthesis 200 will be smaller than the first prosthesis 150 such that the second prosthesis 200 can fit within the bounds of the first prosthesis 150. In other words, a stock implant (the second prosthesis 200) can be entirely or at least partially surrounded by PSI surfaces created by the mold 304.

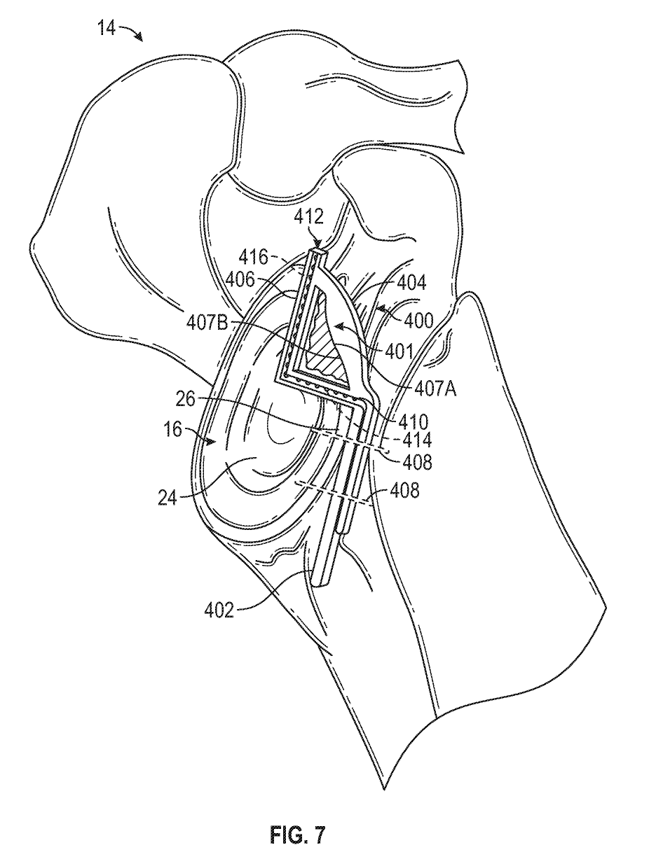

FIG. 7 shows a resection guide 400 fixed to the glenoid 16 near a defect 401 such as a void in the glenoid 16. The defect 401 can occur due to severe wear. The resection guide 400 can be created based on a three-dimensional model of the glenoid 16 similar to the three-dimensional model 22 of FIGS. 2A and 2B. The resection guide 400 can be formed from a material such as plastic, metal or another material or combination of materials. Fabrication of the resection guide 400 can be via machining, molding, and/or additive manufacturing, for example.

The resection guide 400 can include a handle 402, a locating member 404, and a guide member 406. The locating member 404 can engage an irregular surface 207A of the defect 401 to locate the resection guide 400 relative to the glenoid 16. As such, the locating member 404 can include a nesting surface 407B that is configured to mirror and nestingly engage the irregular surface 407B or volume of the defect 401. The nesting surface 407B can be configured such that there is only one way for the resection guide 400 to mate and fit onto the scapula 14. Once located, the resection guide 400 can be fixed to the glenoid 16 using fasteners 408 such as screws. A cutting tool can then be inserted into elongated cutting slots 410, 412 in the guide member 406. The slots 210, 212 can be configured to guide the cutting tool along cut lines 214, 216, respectively. The cutting tool may be inserted into the glenoid 16 at a desired depth using, for example, depth markings.

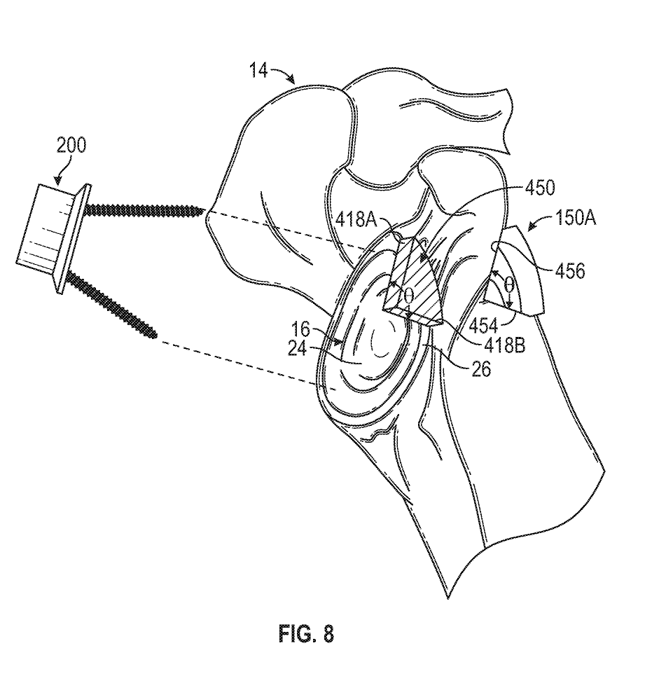

Resection of the glenoid 16 facilitated by the resection guide 400 to remove the defect 401 can then occur leaving linear surfaces 418A, 418B and edges as wells as a void 450 as shown in FIG. 8. The resection guide 400 can then be removed.

A first prosthesis 150A can then be formed either in vivo or with a mold as previously discussed. Thus, the first prosthesis 150A can match the surface of the void 450 and can form a continuous surface with the articular surface surrounding the void 450 according to some examples. As the void 450 has a known shape with substantially smoother surfaces than would otherwise be the case with defect 401 present, the first prosthesis 150A can be constructed to conform to and substantially fill the void 450 more easily. For example, the void 450 and the first prosthesis 150A can have the shape of a wedge, with an angle θ between surfaces 454, 456 of the first prosthesis 150A and between respective surfaces 418A and 418B on the void 450. In some cases, the first prosthesis 150A can have an angle θ that is slightly different than that of the void 450 to exert a pressure on the surfaces 418A and 418B to facilitate integration of the first prosthesis 150A. The second prosthesis 200 can then be implanted over at least a portion of the first prosthesis 150A.

### ADDITIONAL NOTES

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other examples can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above detailed description, various features may be grouped together to streamline the disclosure. This should not be interpreted as intending that an unclaimed disclosed feature is essential to any claim. Rather, inventive subject matter may lie in less than all features of a particular disclosed example. Thus, the following claims are hereby incorporated into the detailed description as examples or embodiments, with each claim standing on its own as a separate example, and it is contemplated that such examples can be combined with each other in various combinations or permutations. The scope of the invention should be determined with reference to the appended claims, along with the full scope of equivalents to which such claims are entitled.

The claimed invention is:

1. A system for treating a defect in a bone that forms at least a portion of a joint of a patient, the system comprising:
a first prosthesis comprising a formable material;
a second prosthesis having a stock construction, the second prosthesis configured to be implanted in the joint on at least a portion of the first prosthesis once the first prosthesis is formed to take a shape of and substantially fill a volume of the defect; and
an alignment guide configured to couple to the second prosthesis to retain the second prosthesis in a desired position and a desired orientation in the joint on the at least the portion of the first prosthesis.

2. The system of claim 1, wherein the first prosthesis comprises one or more of a bone graft, bone, and a synthetic bone putty.

3. The system of claim 1, wherein the first prosthesis in combination with the alignment guide provide for the desired position and the desired orientation of the second prosthesis relative to the joint.

4. The system of claim 1, further comprising a first mold configured to form the first prosthesis, wherein the first mold is formed based on imaging data obtained using a medical imaging technique performed on the joint of the patient, and wherein the first mold includes a cavity that replicates the volume of the defect.

5. The system of claim 4, wherein the first mold includes one or more of a surface and a second cavity configured to replicate an articular surface of the joint.

6. The system of claim 1, further comprising a resection guide configured to position a cutting tool at the joint to perform a resection to remove at least a portion of joint including the defect to create a void with a desired shape in the joint.

7. The system of claim 6, wherein the first prosthesis is configured to substantially take a shape of the void either by insertion into the joint and the void or by being formed in a second mold having a cavity that replicates a volume of the void.

8. The system of claim 1, wherein the alignment guide has one of a stock construction or a patient specific construction.

9. The system of claim 8, wherein the patient specific construction includes a guide surface configured to nest and closely conform to a first surface such that the guide surface mates with the first surface when the alignment guide is in only one orientation.

10. The system of claim 9, wherein the first surface comprises at least one of a surface or an edge of the first mold or the second mold, a surface of the bone that forms the joint, a second surface of the bone that forms the joint the second surface external of the joint, and a surface of a second bone.

11. The system of claim 10, wherein the joint comprises a shoulder and the bone comprises a scapula, and wherein the defect is in a glenoid of the scapula but the first surface comprises a second portion of the scapula external of the glenoid.

12. An assembly for treating a defect in a bone that forms at least a portion of a joint of a patient, the assembly comprising:
a first prosthesis comprising a formable material that is formed to take a shape of and substantially fill a volume of the defect;
a second prosthesis having a stock construction, the second prosthesis implanted in the joint on at least a portion of the first prosthesis; and
an alignment guide connected to the second prosthesis to retain the second prosthesis in a desired position and a desired orientation in the joint on the at least the portion of the first prosthesis.

13. The assembly of claim 12, wherein the first prosthesis comprises one or more of a bone graft, bone, and a synthetic bone putty.

14. The assembly of claim 12, wherein the first prosthesis in combination with the alignment guide provide for the desired position and the desired orientation of the second prosthesis.

15. The assembly of claim 12, wherein the first prosthesis substantially takes the shape of the defect either by insertion into the defect or by being formed in a mold prior to insertion into the joint.

16. The assembly of claim 15, wherein the mold is inserted into the joint and the alignment guide is connected to the mold.

17. The assembly of claim 12, wherein the alignment guide has one of a stock shape or a patient specific shape.

18. The assembly of claim 17, wherein the patient specific shape includes a guide surface configured to nest and closely conform to a first surface such that the guide surface mates with the first surface when the alignment guide is in only one orientation.

19. The assembly of claim 18, wherein the first surface comprises at least one of a surface or an edge of the mold, a surface of the bone that forms the joint, a second surface of the bone that forms the joint the second surface external of the joint, or a surface of a second bone.

20. A method of repairing a defect in a bone that forms at least a portion of a joint of a patient, the method comprising:
receiving imaging data obtained using a medical imaging technique, the imaging data representing the joint of the patient;
creating a three-dimensional model of the joint including the defect based on the imaging data;
implanting a first prosthesis in the joint, the first prosthesis formable to take a shape of and substantially fill a volume of the defect;
implanting a second prosthesis in the joint, the second prosthesis disposed on at least a portion of the first prosthesis; and
coupling an alignment guide to the second prosthesis to retain the second prosthesis in a desired position and a desired orientation in the joint on the at least the portion of the first prosthesis.

21. The method of claim 20, further comprising:
forming a mold based upon the imaging data, the mold including a cavity that replicates the volume of the defect; and
shaping the first prosthesis within the cavity.

22. The method of claim 19, further comprising providing a patient specific guide surface for the alignment guide based upon the imaging data, the guide surface configured to nest and closely conform to a first surface such that the guide surface mates with the first surface when the alignment guide is in only one orientation.

23. The method of claim 22, wherein the first surface comprises at least one of a surface or an edge of the mold, a surface of the bone that forms the joint, a second surface of the bone that forms the joint the second surface external of the joint, or a surface of a second bone.

24. The method of claim 20, further comprising:
creating a three-dimensional model comprising a virtual resection guide based on the imaging data;
fabricating a resection guide configured to guide a cutting tool in resecting the portion of the joint that includes the defect to create a void with a desired shape; and
shaping the first prosthesis to substantially take a shape of the void either by insertion into the joint and the void or by being formed in a second mold having a cavity that replicates a volume of the void.
